(19) United States
(12) Patent Application Publication
(10) Pub. No.: US 2004/0118410 A1
(43) Pub. Date: Jun. 24, 2004

(54) Surgical drape having an instrument holder

(76) Inventors: Henry L. Griesbach III, Clarkston, GA (US); Andrea L. Lewis, Alpharetta, GA (US)

Correspondence Address: DORITY & MANNING, P.A., POST OFFICE BOX 1449, GREENVILLE, SC, 29602-1449, US

(21) Appl. No.: 10/323,305

(22) Filed: Dec. 18, 2002

(51) Int. Cl. A61B 19/08

(52) U.S. Cl. 128/852

(57) ABSTRACT

A surgical drape for use during surgery of a patient is provided. The surgical drape includes a sheet that is configured for covering at least a portion of a patient during surgery. Also included is an instrument holder that is carried by the sheet and is permanently attached to the sheet. The instrument holder is configured for receiving at least one surgical instrument.

### BACKGROUND

Various types of surgical drapes have been used to keep a surgical site on a patient sterile during a surgical procedure. Traditionally, surgical drapes were linen or woven cloth, and were sterilized after each use for reuse. More recently, disposable sterile drapes have been introduced, in which a nonwoven paper or fabric forms a substantial part of the drape. A reinforcement area is often placed around a fenestration or an edge of disposable surgical drapes to provide structural strength and to absorb bodily fluids from a surgical site. Many disposable drapes also include a number of layers of different materials for the drape area and reinforcement area, with each layer providing a different property to the drape. For example, spunbond fabrics, meltblown fabrics, and polymer films have been used as layers in disposable drapes.

Many different shapes of surgical drapes have been proposed, often depending upon the specific surgical procedure to be performed. For example, the shape of the drape is often specifically designed to fit around a specific surgical site on the body. In some cases, a fenestration, as mentioned above, is provided through a drape to allow medical personnel access to the surgical site, whereas the remaining sheet portion of the drape covers the rest of the body and table. Moreover, several drapes are often used in combination as a draping system or kit to cover a patient. In some cases, several rectangular drapes, often called universal drapes, are laid over the patient in a pattern providing an opening through which the medical personnel can access the surgical site white also covering the remainder of the patient's body and the table.

Nevertheless, one problem with such conventional drapes is that various objects and tools cannot be easily positioned on top of the drapes without falling off or without becoming contaminated. In response, some drapes have been developed to maintain medical devices during a surgical procedure. For instance, a prior surgical drape that has been developed for such a purpose includes a plurality of binding strips of material. The binding strips of material are attached to the upper surface of the drape for the purpose of maintaining medical devices thereon during a surgical procedure. The strips may include a fastening system that employs hook and loop type fasteners in order to engage the medical device and retain it thereon. Alternatively, an adhesive strip for sealing the strip onto itself may be employed in order to retain the medical device thereon.

Such retention devices are problematic in that they require a surgeon to manipulate the retention device during surgery in order to fasten the medical device onto the drape. This can be a time consuming and distracting procedure during the course of surgery. Additionally, such fastening systems are undesirable in that they add cost to the drape because the fastening system is a separate component that must be attached to the drape. Being a separate component is also disadvantageous in that it reduces the overall structural integrity of the drape because now the drape is two pieces that are attached to one another.

Additional ways of holding instruments onto drapes may be through instrument pads which are magnetic or have high friction surfaces which prevent instruments from moving. The instrument pads may be re-usable and on different drapes according to the overlaid position. Problems with instrument pads are their high cost, lack of permanent attachment to different drapes, and sterilization requirements distinct from those for disposable sterile drapes.

As such, a need currently exists for a surgical drape that better allows for the positioning or placement of certain medical tools, instruments, etc., thereon without falling off or becoming contaminated.

### SUMMARY

Various features and advantages of the invention will be set forth in the following description, or may be obvious from the description, or may be learned from practice of the invention.

The present invention provides for a surgical drape for use during surgery of the patient that has a sheet that is configured for covering at least a portion of the patient during surgery. Also, an instrument holder is carried by the sheet and is configured for receiving at least one surgical instrument. The instrument holder is permanently attached to the sheet.

In certain exemplary embodiments of the present invention, the instrument holder may contain the same material as the sheet. Further, in other exemplary embodiments at least a portion of the instrument holder may be at a different vertical location with respect to the sheet when the sheet is spread out in a substantially flat configuration. Also, in other exemplary embodiments of the present invention the instrument holder may be thermally molded on the sheet. Alternatively, the instrument holder may be a film that is integral with the sheet and thermoformed into the shape of the instrument holder in other exemplary embodiments.

The instrument holder as discussed above may be configured in various ways in accordance with several different exemplary embodiments of the present invention. For instance, the instrument holder may be a cavity in the surface of the sheet. The instrument holder may have a bottom portion, and sides that completely surround the bottom portion. Alternatively, foam or other resilient material may be present on the drape and used to reinforce the sides and/or the bottom of the instrument holder. Alternatively, the instrument holder may have a bottom portion and at least one side where the side is vertically above the sheet when the sheet is spread out in a substantially flat configuration. In other exemplary embodiments, the instrument holder may be both above or below the sheet. Further, the instrument holder may have a plurality of compartments formed thereon, or may be a single cavity in the surface of the sheet.

Also, the instrument holder may be provided at any location on the sheet. For instance, if a fabric is provided on the sheet in order to absorb fluid which contacts the drape, the instrument holder may be located either in this fabric or spaced therefrom. The instrument holder may be any distance from a fenestration that is provided in the surgical drape. Exemplary embodiments of the present invention include having the instrument holder being located anywhere on the drape.

The present invention also provides for a method of forming the instrument holder in the surgical drape. The method includes positioning the drape between a mold, which may be formed for instance by an embossing plate and a resilient member. The mold used may be, in other exemplary embodiments, any structure that imparts vertical stability to a section of the surgical drape. A force is applied so that the embossing plate engages the drape and at least a portion of the drape is urged into the resilient member. The drape is heated while the drape is being urged into the resilient member and is then removed therefrom. Upon removal from the resilient member, an instrument holder is formed in the drape.

### BRIEF DESCRIPTION OF THE DRAWINGS

FIG. 1 is a perspective view of an exemplary embodiment of a surgical drape in accordance with the present invention. Here, an instrument holder is located in a section of fabric of the surgical drape.

FIG. 1A is a cross section view of the instrument holder taken along line 1A of FIG. 1.

FIG. 1B is a cross section view of another exemplary embodiment of a surgical drape having an instrument holder.

FIG. 2 is a perspective view of an exemplary embodiment of a surgical drape in accordance with the present invention. Here, the instrument holder is located in the sheet portion of the surgical drape.

FIG. 3 is a perspective view of an exemplary embodiment of a surgical drape in accordance with the present invention. Here, the surgical drape is shown as being placed on a patient and having surgical instruments disposed therein.

FIG. 4 is a perspective view of an exemplary embodiment of a surgical drape in accordance with the present invention. Here, the instrument holder is provided with a plurality of compartments for the storage of surgical instruments.

FIG. 4A is a cross section view taken along line 4A of FIG. 4.

FIG. 5 is a perspective view of an exemplary embodiment of a surgical drape kit in accordance with the present invention. Here, one of the surgical drapes is provided with an instrument holder and is connected to a plurality of other surgical drapes that are not provided with instrument holders such that the surgical drapes are connected together via fasteners in order to provide a surgical opening.

FIG. 6 is a perspective view of an exemplary embodiment of a surgical drape in accordance with the present invention. Here, two instrument holders are provided in the surgical drape, and are not rectangular in shape.

FIG. 7 is a perspective view of an exemplary embodiment of a surgical drape in accordance with the present invention. The surgical drape is provided with a deeper instrument holder then those disclosed in previous exemplary embodiments, and the surgical drape has a fabric being present around a fenestration where the instrument holder is spaced from the fabric.

FIG. 7A is a cross section view of the instrument holder taken along line 7A of FIG. 7.

FIG. 8 is a perspective view of an exemplary embodiment of a surgical drape in accordance with the present invention. The surgical drape is provided with an instrument holder that includes a cavity that is vertically below the sheet of the surgical drape when the sheet is spread out in a substantially flat configuration.

FIG. 8A is a cross section view of the instrument holder taken along line 8A of FIG. 8.

FIG. 9 is a perspective view of an exemplary embodiment of a surgical drape in accordance with the present invention. The instrument holder has a bottom portion and at least one side where the side is vertically above the sheet of the surgical drape when the sheet is spread out in a substantially flat configuration.

FIG. 9A is a cross section view of the instrument holder taken along line 9A of FIG. 9.

FIG. 10 is a schematic view of an exemplary embodiment of an embossing procedure in order to form the instrument holder from the drape components such as the fabric and/or the sheet according to the present invention.

FIG. 11 is a perspective view of an exemplary embodiment of a surgical drape in accordance with the present invention. The instrument holder is a pre-formed tray that is attached to the sheet.

FIG. 11A is a cross section view taken along line 11A of FIG. 11.

FIG. 12 is a perspective view of another exemplary embodiment of a surgical drape in accordance with the present invention. The instrument holder contains a foam element that is attached to the sheet.

FIG. 12A is a cross section taken along line 12A of FIG. 12.

### DETAILED DESCRIPTION

As used herein, the terms “nonwoven web” or “nonwoven” refers to a web having a structure of individual fibers or threads which are interlaid, but not in an identifiable manner as in a knitted fabric. Nonwoven webs or fabrics have been formed from many processes, such as, for example, meltblowing processes, spunbonding processes, and bonded carded web processes. The basis weight of nonwoven fabrics is usually expressed in ounces of material per square yard (osy) or grams per square meter (gsm) and the fibers diameters are usually expressed in microns. (Note that to convert from osy to gsm, multiply osy by 33.91).

As used herein, the term “fiber” generally refers to an elongated strand of defined length, such as staple fibers formed by cutting a continuous strand into lengths of, for example, 2 to 5 cm. Collections of fibers may have the same or different lengths.

As used herein, the term “filament” refers toga generally continuous strand that has a large ratio of length to diameter, such as, for example, a ratio of 1000 or more.

As used herein, “meltblown fibers” refers to fibers formed by extruding a molten thermoplastic material through a plurality of fine, usually circular, die capillaries as molten threads or filaments into converging high velocity, usually hot gas (e.g., air) streams which attenuate the filaments of thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al.

As used herein, “spunbond filaments” refer to small diameter filaments that are formed by extruding molten thermoplastic material as filaments from a plurality of fine, usually circular capillaries of a spinneret with the diameter of the extruded filaments then being rapidly reduced as by, for example, in U.S. Pat. Nos. 4,340,563 to Appel et al., 3,692,618 to Dorschner et al., 3,802,817 to Matsuki et al., 3,338,992 to Kinney, 3,341,394 to Kinney, 3,502,763 to Hartman, and 3,542,615 to Dobo et al. Spunbond filaments are generally not tacky when they are deposited on a collecting surface.

As used herein, the phrase “thermal point bonding” generally refers to passing a fabric (e.g., fibrous web or multiple fibrous web layers) to be bonded between a heated calendar roll and an anvil roll. The calendar roll is usually patterned in some way so that the entire fabric is not bonded across its entire surface, and the anvil roll is usually smooth. As a result, various patterns for calendar rolls have been developed for functional as well as aesthetic reasons. One example of a pattern that has points is the Hansen-Pennings or “H&P” pattern with about a 30% bond area with about 200 pins/square inch as taught in U.S. Pat. No. 3,855,046. The H&P pattern has square point or pin bonding areas. Another typical point bonding pattern is the expanded Hansen-Pennings or “EHP” bond pattern that produces a 15% bond area. Another typical point bonding pattern designated “714” has square pin bonding areas wherein the resulting pattern has a bonded area of about 15%. Other common patterns include a diamond pattern with repeating and slightly offset diamonds with about a 16% bond area and a wire weave pattern looking as the name suggests, e.g. like a window screen, with about an 18% bond area. Typically, the percent bonding area varies from around 10% to around 30% of the area of fabric. As is well known in the art, the point bonding holds the resulting fabric together.

It should be noted that any given range presented herein is intended to include any and all lesser included ranges. For example, a range of from 45-90 would also include 50-90; 45-80; 46-89 and the like. Thus, the range of 95% to 99.999% also includes, for example, the ranges of 96% to 99.1%, 96.3% to 99.7%, and 99.91 to 99.999%.

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents.

Surgical drapes formed in accordance with the present invention can generally possess any of a variety of sizes and shapes, depending on the particular use of the drape and on its desired properties. For example, certain surgical drape configurations are described in U.S. Pat. No. 6,055,987 to Griesbach, et al., which is incorporated herein in its entirety by reference thereto for all purposes.

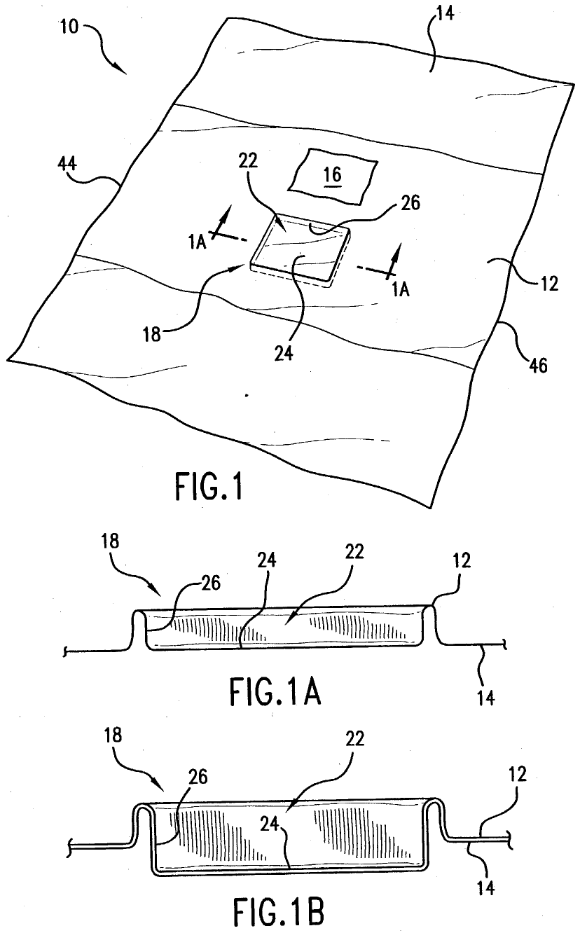

Moreover, in one exemplary embodiment, referring to FIG. 1, a surgical drape 10 having a certain configuration is illustrated. In particular, the surgical drape 10 includes a base sheet 14 to which a portion of a fabric 12 is attached. For example, in one embodiment, the drape 10 contains a 193-centimeter×305-centimeter base sheet 14 made of polyolefinic spunbond and meltblown layers. Moreover, the fabric 12, in one embodiment, has exterior dimensions of 65 centimeters×100 centimeters.

In some embodiments, such as shown in FIG. 1, at least a portion of the fabric 12 is attached to the base sheet 14 using conventional attachment methods, such as thermal point bonding, ultrasonic point bonding, adhesive, or mechanical bonding. In one embodiment, for example, the fabric 12 can be adhesively laminated to the base sheet 14 using an aqueous adhesive, such as an adhesive sold under the name L 8052-01 by Findley Adhesives.

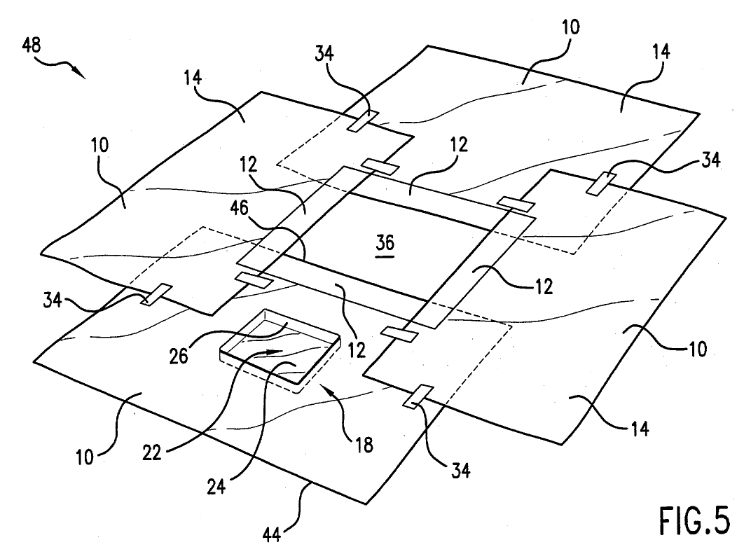

In general, the area of attachment between the fabric 12 and the base sheet 14 can vary. For example, in some embodiments, as shown in FIG. 1, an area less than or equal to the area bounded by the edges 44 and 46 can be bonded to the base sheet 14 using conventional attachment methods. However, while shown in FIGS. 5, it should be understood that the fabric 12 is not necessarily required and, if desired, the base sheet 14 may form substantially the entire surgical drape 10. Moreover, as shown in FIG. 5, an individual fenestration opening is not always required.

As stated, in some embodiments, the drape 10 includes a fenestration opening 16 that can be placed over an operating site during surgery. For example, in one embodiment, a 10-centimeter×30.5-centimeter fenestration opening 16 is provided. In the embodiment depicted in FIG. 1, the fabric 12 also surrounds each side of the fenestration opening 16 so that it may absorb fluids therefrom. However, in some embodiments, the fabric 12 can be placed adjacent to only one, two, or three sides of the fenestration 16.

If desired, the fabric 12 may be constructed so as to have properties that differ from the base sheet 14. For example, large-sized drapes that are intended to completely cover the patient and provide substantial fluid absorption can use an absorbent multilayered nonwoven fabric 12 surrounding the fenestration 16. A film as one of the layers may be used to inhibit the passage of fluids through the drape 10. The fabric 12 may be used to provide structural support in the area surrounding the fenestration 16. Moreover, it should be understood that the surgical drape 10 of the present invention need not contain a separate base sheet 14. Moreover, as stated above, it should be understood that the surgical drape 10 of the present invention need not contain a fabric 12.

In another embodiment, such as shown in FIG. 5, a drape kit 48 is provided that includes a plurality of surgical drapes 10 each formed from a base sheet 14 and having a fabric 12. For example, in one embodiment, the drape kit 48 includes at least one fastener 34 for securing at least two drapes 10 together. The fastener 34 may be adhesive strips or hook fasteners or mechanical fasteners (e.g. clips) in other exemplary embodiments of the present invention. As specifically shown in FIG. 5, four drapes 10 and eight hook fasteners 34 are provided. Specifically, each drape 10 is secured to two other drapes 10 via two fasteners 34. However, it should be understood that the drapes 10 can be attached to each other using other methods as well.

The drape kit 48 may include any number of drapes 10 greater than one, and any number of fasteners 34. As shown, the four drapes 10 are arranged so as to define a surgical opening 36 between the drapes 10. Such an opening 36 could be created by medical personnel for performing a surgical technique on a patient without the need for a specifically designed drape 10. Thus, the drapes 10 of the drape kit 48 could be arranged in any desired manner over the top of a patient and operating table to provide much greater flexibility to the medical personnel.

Various embodiments of the materials and methods used to construct a surgical drape 10 in accordance with the present invention will now be described in more detail. In general, the fabric 12 may be made from the same or different materials as the base sheet 14. For example, the fabric 12, as discussed in more detail below, may be constructed from nonwoven layers, adhesive layers, film layers, magnetic layers, etc. Moreover, some or all of the fabric 12 may be constructed so as to be hydrophilic or hydrophobic, and may be chemically treated to achieve the desired absorbency properties. For instance, the fabric 12 and/or one or more layers of the fabric 12 may be treated with a surfactant in a manner such as described in U.S. Pat. No. 5,540,979, which is incorporated herein in its entirety by reference thereto for all purposes.

The fabric 12 may in another exemplary embodiment of the present invention be a spunbond layer attached to a middle layer of a meltblown material which is further attached to a backing layer being a fluid impervious film. This type of an arrangement for the fabric 12 allows for the reinforcement of the area adjoining the fenestration 16 allows for fluid absorption, and ensures a fluid impervious barrier. Construction of such a fabric 12 is described in U.S. Pat. No. 4,379,192 to Wahlquist et al. which is incorporated by reference herein in its entirety for all purposes. The absorbent properties present in the spunbond layer and to some extend the film may be polymers and treatments that are described in U.S. Pat. No. 5,540,979 to Yahiaoui et al. which is incorporated by reference herein in its entirety for all purposes. A suitable fabric 12 having a spundbound-meltblown-film arrangement is Control® Plus manufactured by Kimberly-Clark located at 1400 Holcomb Bridge Road, Roswell, Ga. 30076-2199.

As stated, the fabric 12 may be multilayered and may include a nonwoven layer disposed on one surface of the fabric 12. The nonwoven layers can generally have a variety of basis weights. For example, in one embodiment, the layers have a collective basis weight of about 64 grams per square meter. In addition, the nonwoven layers can be formed in a variety of ways and can be formed from a variety of different materials. For example, in one embodiment, one surface layer is a spunbonded fabric containing fabrics and/or filaments having diameters of about 2.0 denier and made from a copolymer of propylene with 3.5% ethylene (e.g., Union Carbide 6D43 from Union Carbide Corp.) having a basis weight of 20 grams per square meter. Moreover, in this embodiment, the spunbound layer is bonded by a repeating pattern of discretely fused areas spaced apart to give 111 fused areas per square inch and a 17.7% total bonded area.

In another embodiment, the nonwoven layer of the fabric 12 is a spunbond fabric formed from two layers of multicomponent filaments, such as described in U.S. Pat. No. 5,418,045, which is incorporated herein in its entirety by reference thereto for all purposes. In particular, the spunbonded fabric is bonded so that a plurality of point unbonded circular areas of about 0.4 centimeters are spaced approximately 0.5 centimeters apart in a hexagonal (close packing) arrangement. The upper surface of the spunbonded fabric may be composed of fibers and/or filaments having a diameter of about 9.0 denier (e.g., circular in cross-section and have polymeric components of Exxon 3445 polypropylene and Dow ASPUN 6811A linear low density polyethylene arranged in a side-by-side configuration at 49% by weight with the remainder being titanium dioxide pigment). The lower surface of the spunbonded fabric may be composed of filaments that have a diameter of about 2.0 denier and have the same composition as the 9-denier filaments mentioned above. Moreover, in one embodiment, the spunbonded fabric is bonded by a repeating pattern of discretely fused areas spaced apart to give a total bond area of about 15%.

Fabric 12 may also be of a single nonwoven layer, without additional layers, as is suitable when base sheet 14 contains a film to impart fluid barrier attributes and fabric 12 needs only to impart enhanced absorbency.

A variety of lamination techniques can also generally be utilized to laminate the layers of the fabric 12 to one another. For example, in one embodiment, a film layer of the fabric 12 is initially provided with a thickness of between about 0.0010 to about 0.0015 inches. The layers can then be laminated using conventional techniques. For example, in one embodiment, meltblown layers can be thermally laminated to spunbond layers using discrete bond points, such as described in U.S. Pat. No. 5,540,979 to Yahiaoui et al.

Although not required in addition to the nonwoven layers in fabric 12, additional layers may be attached above or below the nonwoven layers in the bottom portion of an instrument holder 18. Such layers can impart attributes other than these of the nonwoven layers, for instance, magnetic attributes, slip resistant, cut resistant, etc.

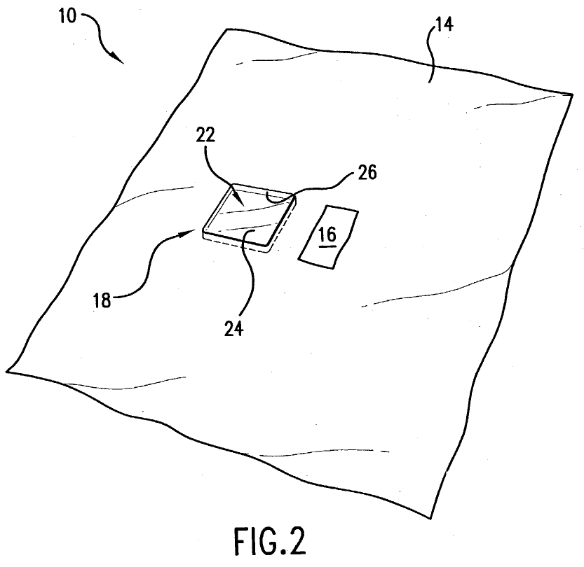

The drape 10 in accordance with the present invention has an instrument holder 18 as shown in FIG. 1 that is carried by the drape 10. The instrument holder 18 is permanently attached to the drape 10. In one exemplary embodiment, a mold may be used in order to make the instrument holder 18 integrally formed with the drape 10. The instrument holder 18 may be formed in the fabric 12 of the drape 10 as shown in FIG. 1, or may be formed in the sheet 14 of the drape 10 as shown in FIG. 2. Additionally, the instrument holder 18 may be a separate piece that is attached to the sheet 14 or fabric 12. As such, the present invention is not limited to having the instrument holder 18 being formed in a particular surface, layer, or location of the drape 10. For instance, the instrument holder 18 may be located preferably within the sterile field such that it is substantially horizontal in orientation. Referring to FIG. 1, the instrument holder 18 includes a cavity 22 that is distinct from the sheet 14 when the sheet 14 is spread out in a substantially flat configuration, for instance when the sheet 14 is held by two people on opposite ends with no overlap of any portion of the sheet 14 and with the cavity 22 being accessible to a clinician. This can be seen in better detail in FIG. 1A where the cavity 22 is shown as being vertically below and above the surface of the sheet 14. In one exemplary embodiment of the present invention, the cavity 22 of the instrument holder 18 is approximately 1cm in depth. The instrument holder 18 may be placed adjacent or in close proximity to the fenestration 16 so that the instrument holder 18 is located in or substantially in a sterile field during surgery. FIG. 1B shows more clearly the fabric 12 being a separate layer from the sheet 14.

Hands-free passing is a technique in which a sharp surgical instrument such as a needle or a sharp is placed on a surface by a caregiver and then picked up from that surface by another caregiver. In essence, the sharp is not passed from one hand of a medical caregiver to another hand of another medical caregiver. The hands free technique minimizes the likelihood that medical caregivers touch the same instrument at the same time. This technique can be accomplished by laying the sharp down into a neutral zone such as the instrument holder 18 of the invention to be subsequently retrieved by a separate medical caregiver.

A recent study conducted by the University of British Columbia at Vancouver found that the effectiveness of the hands-free passing technique reduced the incidence of sharp injuries by 60% in procedures in which significant blood loss occurs. The researcher concluded that the use of the hands-free technique is effective in reducing the incidence of percutaneous injuries, glove tears, and contamination of the sterile field during surgery. The present invention allows for the use of the hands-free passing technique in that the medical caregiver may lay the instrument into the instrument holder 18 from which it may be retrieved at a later time by a different medical caregiver. The instrument holder 18 may therefore be thought of as a neutral zone or thought of as being located within a neutral zone.

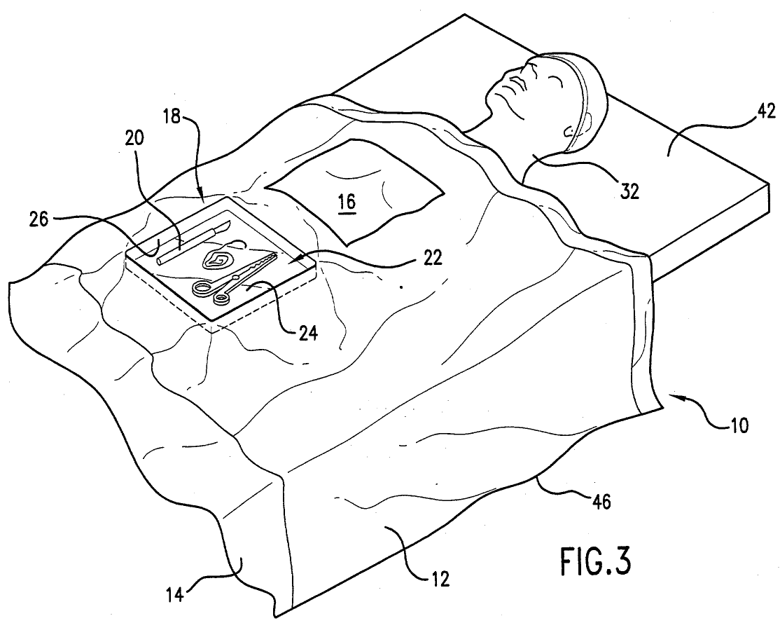

FIG. 3 shows an exemplary embodiment of the drape 10 in accordance with the present invention being placed on a patient 32 who is located on an operating table 42. In this particular embodiment, the drape 10 lays over an edge of the operating table 42. The instrument holder 18 is configured for receiving and holding a plurality of surgical instruments 20. Various objects such as the surgical instruments 20 may be positionable and repositionable in a variety of ways within the instrument holder 18. A few of the objects and surgical instruments 20 that may be held within the instrument holder 18 in accordance with the present invention include, but are not limited to, fiber optical cords, endoscopic tubing, cords for pencils, smoke evacuator tubing, irrigation/aspiration tubing, sharps and the like. The instrument holder 18 provides an area where the surgical instruments 20 can be placed when not in use. The instrument holder 18 prevents the surgical instruments 20 from sliding off of the sterile field present during surgery and establishes a visible neutral zone for use in hands free passing techniques. This prevents confusion about location of the instruments 20 and then presents them for reuse during the surgical procedure, in effect increasing the speed and/or safety at which a surgical procedure is preformed.

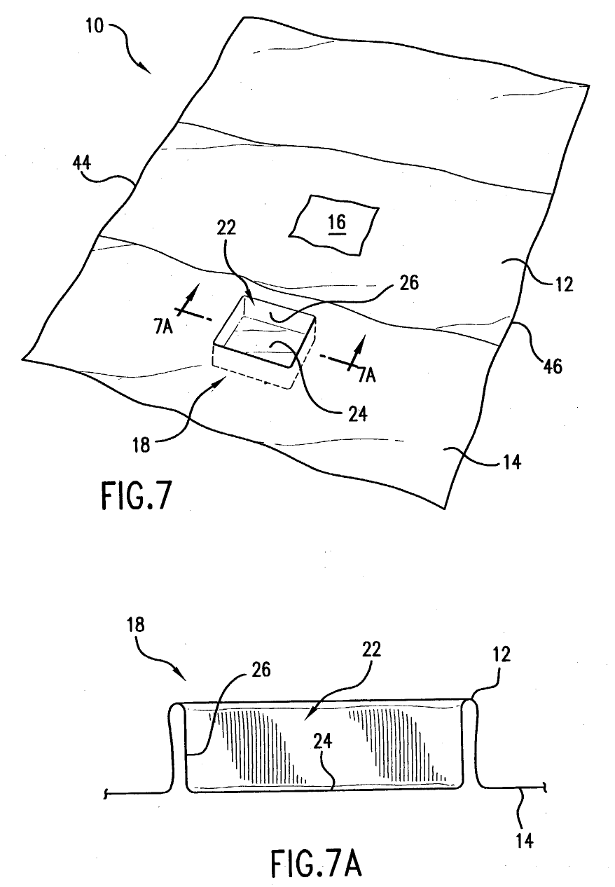

Although shown as being located next to the fenestration 16 in FIG. 1, it is to be understood that in other exemplary embodiments of the present invention that the instrument holder 18 may be located on other portions of the drape 10 away from the fenestration 16. For instance, FIG. 7 shows one exemplary embodiment of the present invention where the instrument holder 18 is located in the drape 10 at a location removed from the fenestration 16. Additionally, the cavity 22 of the instrument holder 18 is approximately 3 cm in depth, this being a greater depth than the instrument holder shown in FIG. 1. Also, the instrument holder 18 in FIG. 7 is shown as being formed within the sheet 14 instead of the fabric 12. As previously mentioned, the invention is not limited to having the instrument holder 18 being located at a particular location or formed in a particular material of the drape 10. For instance, the present invention includes exemplary embodiments where the drape 10 is provided with the sheet 14 but not the fabric 12. This exemplary embodiment is shown in FIG. 2 of the drawings. Here, the instrument holder 18 is formed in the sheet 14 and is located proximate to the fenestration 16.

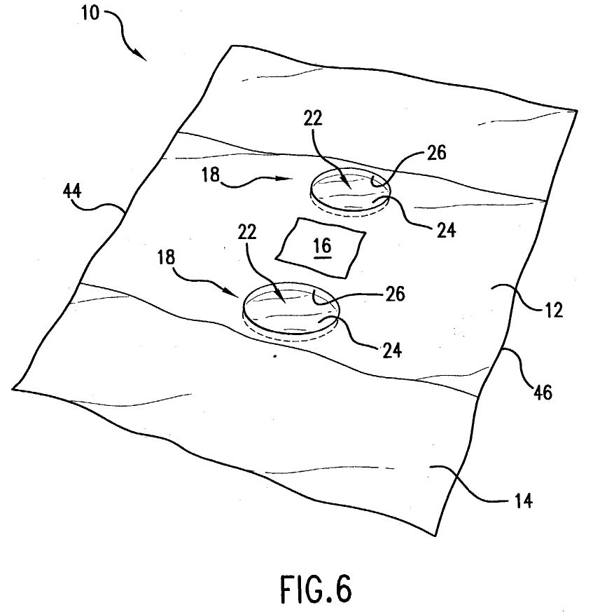

Although shown as being a rectangular cavity 22 in FIG. 1, the instrument holder 18 may be formed having a shape other than a rectangular cavity 22. For instance, FIG. 6 shows an exemplary embodiment of the present invention where the instrument holder 18 is a circular cavity in the fabric 12. Additionally, a pair of instrument holders 18 are shown in the exemplary embodiment of FIG. 6. As such, the present invention includes other exemplary embodiments where any number of instrument holders 18 are located on the drape 10, and is not limited to embodiments only having a single instrument holder 18 located on the drape 10. As can be seen in FIG. 6, the pair of instrument holders 18 are located proximate to the fenestration 16 and are within the sterile field of the drape 10 once placed upon a patient and in a functional position for a neutral zone.

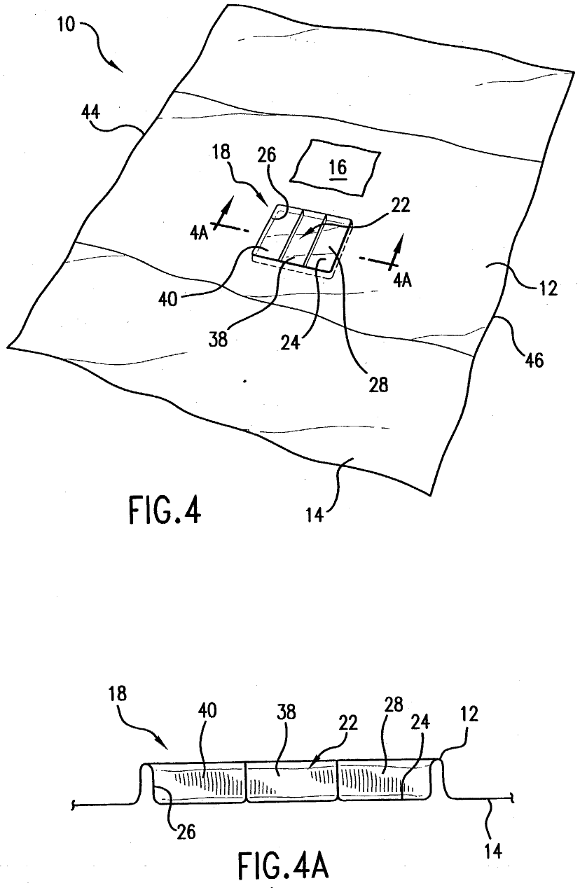

Another alternative exemplary embodiment of the present invention is shown in FIG. 4. Here, the instrument holder 18 is located on the fabric 12 of the drape 10 proximate to the fenestration 16. The instrument holder 18 shown is provided with a series of compartments 28, 38, and 40 within the cavity 22. The compartments 28, 38, and 40 may be of any shape or size, although shown in FIG. 4 as being rectangular in shape and equal to one another in size. Providing the instrument holder 18 with several different compartments 28, 38, and 40 may provide for more desirable storage and holding of the surgical instruments 20 and other objects. Also, the compartments 28, 38, and 40 may be designed so that they may accommodate differently sized and shaped surgical instruments 20 and other objects. The portions of the instrument holder 18 that form the compartments 28, 38, and 40 may also be made of the fabric 12, or other parts of the drape 10 from which the instrument holder 18 is formed.

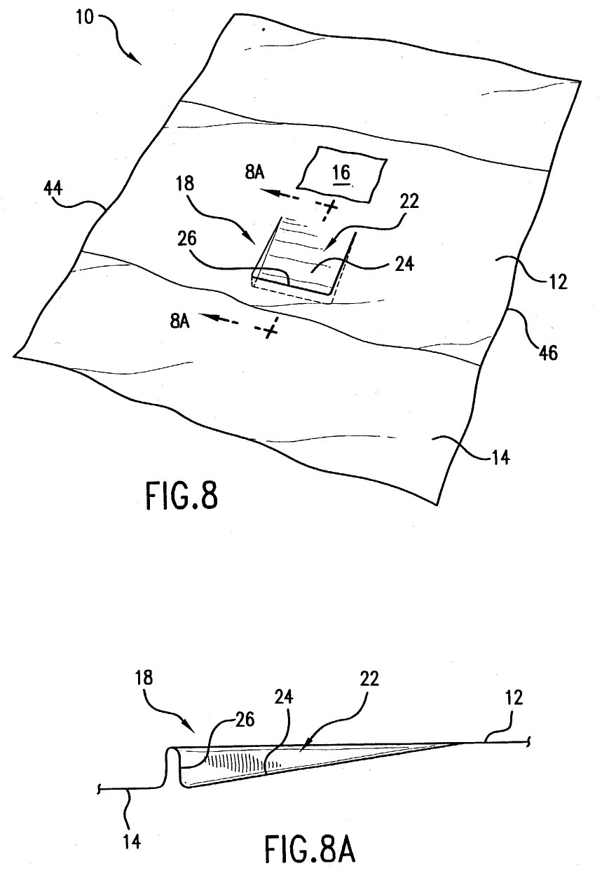

An additional exemplary embodiment of the present invention is shown in FIG. 8. Here the instrument holder 18 is provided with a side 26 that defines a partial cavity 22 where the side 26 extends vertically above the surface of the sheet 14 when the sheet 14 is spread out in a substantially flat configuration, for instance when two people hold opposite ends of the sheet 14, as shown, for instance, in FIG. 8A. Providing the side 26 above the surface of the sheet 14 allows for the instrument holder 18 to be formed having a partial cavity bottom portion 24 that slopes downwardly from the surface of the drape 10 to the bottom of the side 26. The bottom portion 24 is made of the same material as the fabric 12 or sheet 14 into which the instrument holder 18 is located. It is therefore the case that the bottom portion 24 of the instrument holder 18 does not have to be a substantially flat surface, as shown for instance in FIGS. 1, 2, and 7, or bounded by shoulders on all sides to define an enclosing perimeter.

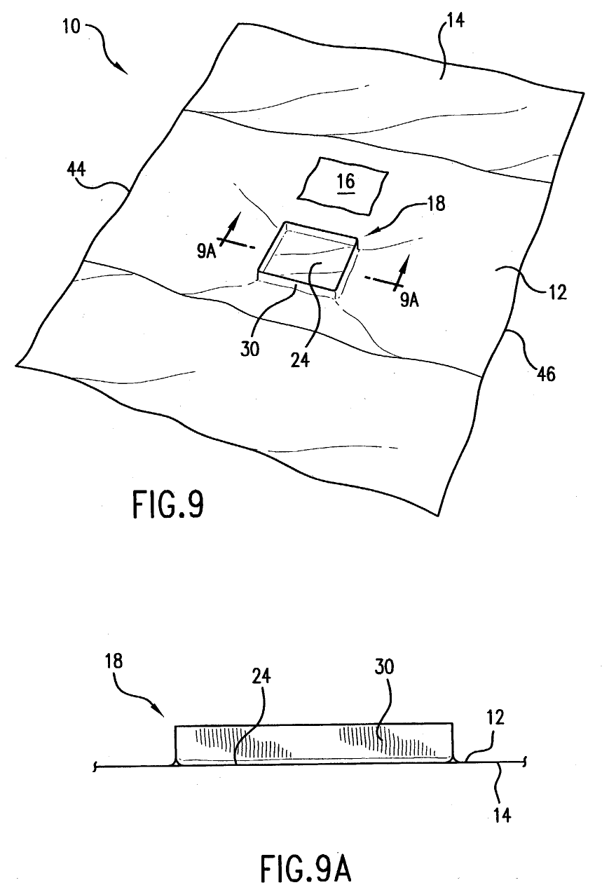

Referring back to FIG. 1, the instrument holder 18 is provided with four sides 26 that are above the surface of the drape 10 when the drape 10 is spread out in a substantially flat configuration. This can be seen, for instance, in FIG. 1A which shows the side 26 being above the upper surface of the fabric 12 and the sheet 14. However, it is to be understood that the sides 26 of the instrument holder 18 are integral in this exemplary embodiment with the fabric 12 and/or the sheet 14 and are not separate components. FIG. 9 shows such an embodiment where the instrument holder 18 is provided with four sides 30 that are vertically above the fabric 12 and the sheet 14 when the fabric 12 and the sheet 14 are spread out in a substantially flat configuration. Here, the instrument holder 18 is rectangular in shape and is enclosed by the sides 30. Surgical instruments 20 or other objects may be placed within the instrument holder 18 and prevented from leaving therefrom by the sides 30, in effect allowing for the use of the hands-free passing technique. The sides 30 are integral with the fabric 12, and also the bottom portion 24 of the instrument holder 18 is integral with the fabric 12.

As can be seen, the instrument holder 18 is capable of being made in various shapes and sizes and may be located at various locations on the drape 10. For instance, the instrument holder 18 may be a closed type instrument holder as the ones shown in FIGS. 1, 7, and 9, or alternatively may be an opened type instrument holder as the one shown in FIG. 8 where the instrument holder 18 does not have sides 26 completely surrounding the instrument holder 18.

The instrument holder 18 may be molded on the sheet 14 and/or fabric 12 so as to be integrally formed on the sheet 14 and/or fabric 12. By being integrally formed, the instrument holder 18 is formed so as to be nonremovable and permanently attached to the drape 10. In effect, the instrument holder 18 is part of the drape 10 as opposed to being a separate component that is overlaid on the drape 10. Also, when described as being “below” and “above” the drape 10, the instrument holder 18 may be described as being “above” or “below” when the drape 10 is spread out in a substantially flat configuration. Here, a portion of the instrument holder 18 may be located vertically above or below the sheet 14 when spread out by two people holding opposite ends of the sheet 14. Although the instrument holder 18 is described as being either “above” or “below” the drape 10, it is to be understood that in some exemplary embodiments of the present invention that the instrument holder 18 may be at the same time “above” and “below” the drape 10. This could be, for instance, when the instrument holder 18 is provided with the cavity 22 as shown in FIG. 1B and is also provided with the vertical sides 30 as shown in FIG. 9.

The instrument holder 18 may therefore be made of the same material as the drape 10. For instance, the instrument holder 18 may be made out of the same fabric 12 that is positioned on the drape 10 proximate to the fenestration 16. As such, the instrument holder 18 may possess the same properties as the fabric 12 or sheet 14 from which it is formed. In this manner, if the fabric 12 is capable of absorbing fluid that is spilled onto the drape 10, the instrument holder 18 is also capable of absorbing the fluid once contacted. Additionally, a magnetic strip may be placed under a nonwoven layer in order to further hold instruments 20 in other exemplary embodiments.

Several different ways of forming the instrument holder 18 in accordance with different exemplary embodiments of the present invention may be utilized. For instance, an object may be placed against the drape 10 which is rectangular in shape: The portion of the drape 10 surrounding the object may then be heat set using heat and pressure so that the instrument holder 18 is formed. In one exemplary embodiment, the fabric 12 may be made from a thermoplastic or thermoset polymer that is capable of accepting a heat set.

An alternative way of making the instrument holder 18 would be to attach a sheet of rigid or semi-rigid thermoplastic/set foam or film above, within, or below the fabric 12 or the sheet 14. This foam or film could then be thermoformed into the shape of the instrument holder 18 either before or after attaching the formed holder to the drape 10. Depending upon the type of foam or film that is used, the foam or film may be attached to the drape 10 using adhesive, ultrasonic welding, or may be attached during the thermoforming tray shaping process. The foam or film may be an integral part of the drape 10 making the instrument holder 18 integrally formed on the drape 10.

Additionally, the instrument holder 18 may be integrally formed on the drape 10 by being treated with a curable chemical in order to harden the section of the drape 10 that is formed into the instrument holder 18 such as a moisture or ultraviolet cross-linking coating

The instrument holder 18 may be formed in the fabric 12 and/or sheet 14. As used in the claims, the term “sheet” is broad enough to cover any portion of the drape 10, and not just those portions separate from the fabric 12. The word “sheet” as used in the claims covers any portion of the sheet 14 and/or the fabric 12.

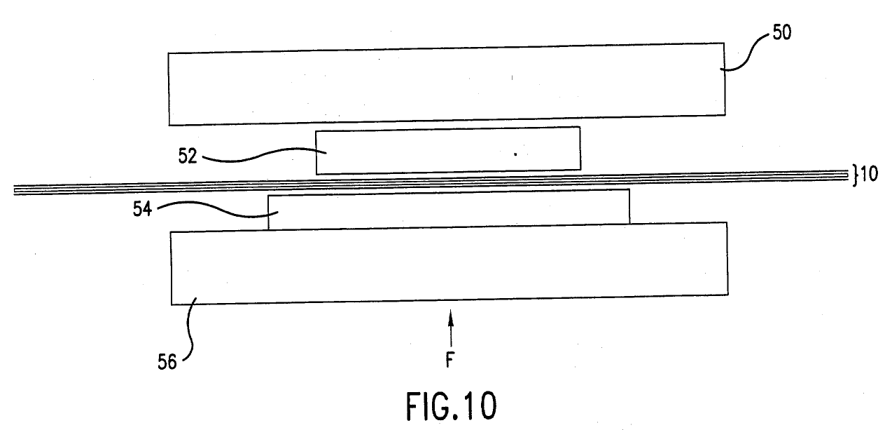

The instrument holder 18 may also be formed on the drape 10 through a thermal embossing method. The thermal embossing method may be done such that excessive densification of the fibrous components such as the spunbond and/or meltblown layers of the drape 10 are avoided. Also, the thermal embossing method may be done such that fusing of the fabric 12 onto a film backing that may be present in the drape 10 does not occur. The thermal embossing method includes providing an unfolded portion of the drape 10 as shown in FIG. 10 that is disposed between a mold, for instance a resilient member 54 and an embossing plate 52. In one exemplary embodiment of the present invention, a fixed plate 50 is located adjacent to the embossing plate 52 and a base 56 is located adjacent to the resilient member 54.

A force shown in the direction of arrow F in FIG. 10 is applied to the base 56 which presses the embossing plate 52 into the drape 10 and into the resilient member 54. The embossing plate 52 may be heated to a desired temperature by the fixed plate 50. Using an appropriate amount of force F, temperature, and time under pressure, the instrument holder 18 may be imparted into the drape 10.

The resilient member 54 may be foam that is high temperature resistant silicone rubber that is deformable and completely recovers to its original thickness after compression. In certain exemplary embodiments of the present invention, the resilient member 54 may be foam that has a thickness of 1.905 cm, and in other exemplary embodiments 2.86 cm.

Also, in one exemplary embodiment of the present invention the embossing plate 52 may be heated to a set point temperature of 145° F. The temperature range of the embossing plate 52 may be between 128° F. and 152.5° F. The fabric 12 used may be Control® Plus. Also, the time of the compressive force is set at two minutes, and the pressure that is applied to the base plate 56 is 20 psi. Higher temperatures and/or longer time durations or conducting the embossing without the resilient member 54 may in some exemplary embodiments cause the fiber and/or film components of the drape 10 to fuse partially or completely rather than simply being densified. If this occurs, fluid imparted onto the instrument holder 18 will not be absorbed by the drape 10. This situation is also encompassed under the scope of the present invention.

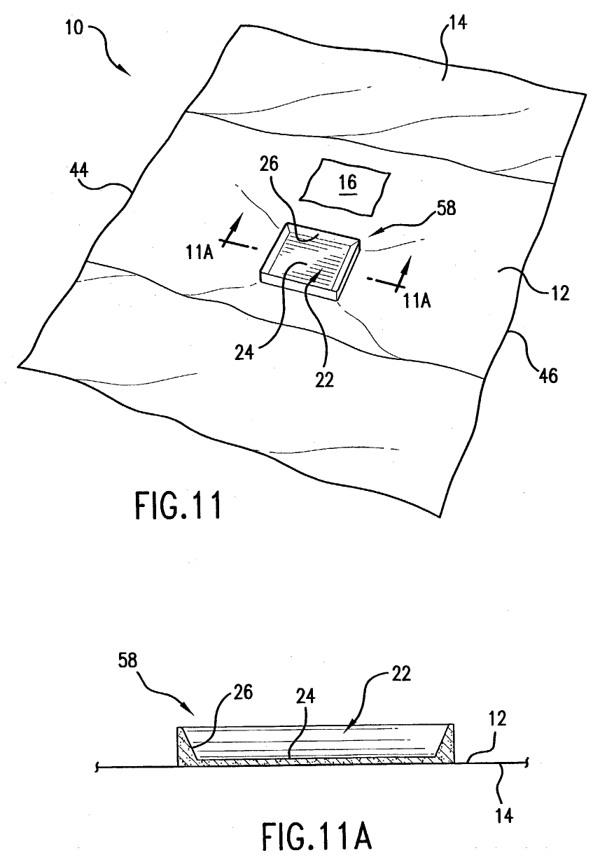

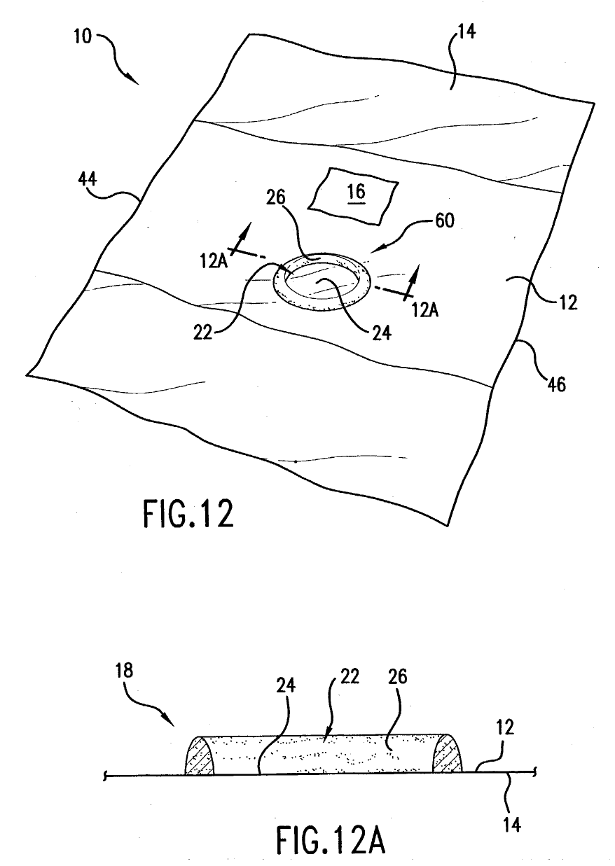

Although described as being integrally formed on the drape 10, the instrument holder 18 in accordance with the present invention may also be a separate piece that is attached to the drape 10. The foregoing description of the instrument holder 18 being integrally formed with the sheet 14 is only an exemplary embodiment of the present invention. For instance, FIG. 11 shows a pre-formed tray 58 that is attached to the fabric 12 of the drape 10. The pre-formed tray 58 may be a rigid piece, a semi-rigid piece, or of the same flexibility as the rest of the drape 10. The preformed tray 58 may be attached to the drape 10 through a variety of means commonly known in the art. For instance, the preformed tray 58 may be attached through adhesion, thermo-ultrasonic welding for permanent attachment, either on the surface or below, or within the fabric 12 or sheet 14. FIG. 12 shows another exemplary embodiment of the present invention where the instrument holder 18 is a separate element that is attached to the surface of the drape 10. Here, the instrument holder 18 is a foam element 60 that is attached to the surface 12. As such, the present invention includes exemplary embodiments where the instrument holder 18 is a separate piece that is attached to the drape 10 and exemplary embodiments where the instrument holder 18 is formed integrally with the drape 10.

It should be understood that the present invention includes various modifications that can be made to the exemplary embodiments of the surgical drape described herein as come within the scope of the appended claims and their equivalents.

What is claimed:

1. A surgical drape for use during surgery of a patient, comprising:
a sheet configured for covering at least a portion of the patient during surgery; and
an instrument holder carried by said sheet, said instrument holder permanently attached to said sheet and defining a cavity in the surface of said sheet, said instrument holder being configured for receiving at least one surgical instrument.

2. The surgical drape of claim 1, wherein said instrument holder is integrally formed with said sheet.

3. The surgical drape of claim 2, wherein said instrument holder is thermally molded in said sheet.

4. The surgical drape of claim 2, wherein said instrument holder is made of the same material as said sheet.

5. The surgical drape of claim 1, wherein said instrument holder comprises a bottom portion and sides completely surrounding said bottom portion.

6. The surgical drape of claim 1, wherein said instrument holder comprises a bottom portion and at least one side, said at least one side being vertically above said sheet when said sheet is spread out in a substantially flat configuration.

7. The surgical drape of claim 1, wherein said instrument holder comprises a plurality of compartments.

8. The surgical drape of claim 1, wherein said sheet has a fenestration and a fabric surrounding said fenestration, said fabric providing for substantial fluid absorption, said instrument holder being located in said sheet and spaced from said fabric.

9. The surgical drape of claim 1, wherein said sheet includes a fabric, said fabric providing for substantial fluid absorption, said instrument holder being located in said fabric.

10. The surgical drape of claim 1, wherein said instrument holder is rectangular in shape.

11. The surgical drape of claim 1, wherein said instrument holder is a film integral with said sheet and thermoformed into the shape of said instrument holder.

12. The surgical drape of claim 9, wherein said instrument holder is formed of a foam element between the sheet and fabric.

13. The surgical drape of claim 1, wherein said instrument holder is a pre-formed tray attached to said sheet.

14. The surgical drape of claim 1, wherein said instrument holder is a foam element attached to said sheet.

15. A surgical drape for use during surgery of a patient, comprising:
a sheet configured for covering at least a portion of the patient during surgery; and
an instrument holder permanently carried by said sheet, said instrument holder being configured for receiving at least one surgical instrument.

16. The surgical drape of claim 15, wherein said instrument holder is molded so as to be integrally formed with said sheet.

17. The surgical drape of claim 16, wherein said instrument holder is thermally molded in said sheet.

18. The surgical drape of claim 16, wherein said instrument holder is made of the same material as said sheet.

19. The surgical drape of claim 15, wherein said instrument holder includes a cavity defined by sides vertically above said sheet when said sheet is spread out in a substantially flat configuration.

20. The surgical drape of claim 15, wherein said instrument holder comprises a bottom portion and sides completely surrounding said bottom portion.

21. The surgical drape of claim 15, wherein said instrument holder comprises a bottom portion and at least one side, said at least one side being vertically above said sheet when said sheet is spread out in a substantially flat configuration.

22. The surgical drape of claim 15, wherein said instrument holder comprises a plurality of compartments.

23. The surgical drape of claim 15, wherein said sheet has a fenestration and a fabric surrounding said fenestration, said fabric providing for substantial fluid absorption, said instrument holder being located on said sheet and spaced from said fabric.

24. The surgical drape of claim 15, wherein said sheet includes a fabric, said fabric providing for substantial fluid absorption, said instrument holder being located in said fabric.

25. The surgical drape of claim 15, wherein said instrument holder is rectangular in shape.

26. The surgical drape of claim 15, wherein said instrument holder is a film integral with said sheet and thermoformed into the shape of said instrument holder.

27. The surgical drape of claim 15, wherein said instrument holder has a magnetic layer embedded in the said sheet.

28. The surgical drape of claim 15, wherein said instrument holder is a pre-formed tray attached to said sheet.

29. The surgical drape of claim 15, wherein said instrument holder is a foam element attached to said sheet.

30. A surgical drape for use during surgery of a patient, comprising:
a sheet configured for covering at least a portion of the patient during surgery, said sheet having a fenestration to allow access to a surgical site through said sheet; and
an instrument holder thermally molded and carried by said sheet, said instrument holder being made of the same material as said sheet and being integral with said sheet, said instrument holder being configured for receiving at least one surgical instrument.

31. A method for forming an instrument holder in a surgical drape, comprising:
positioning a drape between a embossing plate and a resilient member;
applying a force such that said embossing plate engages said drape and at least a portion of said drape is urged into said resilient member;
heating said drape while said drape is being urged into said resilient member; and
removing said shape from said resilient member, an instrument holder being formed in said drape.

32. The method of claim 31, wherein said resilient member is a high temperature resistant rubber deformable foam.

33. The method of claim 31, further comprising:
providing a fixed plate adjacent to said embossing plate;
providing a base adjacent to said resilient member; and
wherein said step of applying a force includes applying a force against said base such that said embossing plate and at least a portion of said drape is urged into said resilient member.

34. The method of claim 31, wherein said drape is heated to a temperature of at least 145° F., and said step of applying force occurs for at least two minutes.
